(19) United States
(12) Patent Application Publication
(10) Pub. No.: US 2024/0189559 A1
(43) Pub. Date: Jun. 13, 2024

(54) FLUID APPLICATOR DEVICE, AND SYSTEMS AND METHODS RELATING THERETO

(71) Applicant: Christian K. Johnson, Los Angeles, CA (US)

(72) Inventor: Christian K. Johnson, Los Angeles, CA (US)

(21) Appl. No.: 18/379,375

(22) Filed: Oct. 12, 2023

Related U.S. Application Data
(63) Continuation of application No. PCT/US22/25144, filed on Apr. 16, 2022.
(60) Provisional application No. 63/175,658, filed on Apr. 16, 2021.

(51) Int. Cl. A61M 35/00

(52) U.S. Cl. CPC A61M 35/003; A61M 2209/082

(57) ABSTRACT

Systems, devices and methods are provided for the use of an applicator device for applying fluid, such as a topical substance, to the body, detecting anomalies, and collecting, processing and displaying data related to monitored skin data and trend information. In particular, described herein are various embodiments of applicator devices, and components thereof, designed to improve a user's ability to apply topical substances to hard-to-reach areas and in a manner so as to provide complete and adequate coverage.

### CROSS-REFERENCE TO RELATED APPLICATIONS

The present application is a continuation of International Patent Application No. PCT/US22/25144, filed Apr. 16, 2022, which claims priority to U.S. Provisional Patent Application No. 63/175,658, filed on Apr. 16, 2021, the entire contents and disclosures of both of which are hereby incorporated by reference.

### BACKGROUND

Excess exposure to sunlight and ultraviolet (UV) radiation can cause short-term and long-term skin damage, such as, e.g., sunburns, sun blisters, sun poisoning, premature skin aging, and even skin cancer. Over three million people are diagnosed with skin cancer annually in the United States. Nearly ninety percent of skin aging and skin cancers are associated with sun exposure and sunburns. A person's risk of melanoma, a type of skin cancer, doubles with either a minimum of five sunburns or one blistering sunburn. The risk of developing melanoma increases by eighty percent for individuals who experience sunburns during the ages of fifteen to twenty-five. If metastasized, melanoma has a near seventy-five percent mortality rate.

Melanoma, however, can be prevented. Consistent daily use of SPF 15 sunscreen is shown to reduce a person's risk of melanoma by fifty percent and reduce signs of skin aging by twenty-four percent. Many people resort to the use of tanning oils, creams, lotions, and sunscreens to block the harmful UV rays that produce the aforementioned ailments. As it relates to skin products used to protect against harmful UV exposure, many users apply said products by hand onto the body. While these products are effective in providing UV protection, they require that an adequate layer be applied over all exposed skin surfaces. While one can usually apply the product over the majority of the body, coverage of hard-to-reach areas, such as one's back, is difficult and usually requires the aid of another person.

Topical substances in the form of an aerosol spray pose an additional difficulty. Aerosol particles can disperse in the air, or with the wind, if not applied directly to the body, thus, further impeding a user's ability to effectively protect their skin. Unfortunately, many people will not know that coverage was inadequate until it is too late and damage has already accrued on the skin, e.g., a sunburn, mole, lesion, etc. Moreover, most people are not trained to conduct a monthly self-screen skin exam or know how to distinguish between a variety of skin lesions (e.g., bites, rash, burn, freckle, mole, pre-cancerous, cancerous, etc.), and determine whether a skin development is risky or dangerous. Lack of awareness or certainty can prevent an individual from identifying and/or scheduling an appointment with a physician, leading them to wait and see if the lesion resolves on its own, potentially leading to catastrophic results.

Therefore, it would be advantageous to provide devices, systems, and methods capable of applying topical fluids and/or substances to the back region and other hard-to-reach areas easily so as to provide complete and adequate coverage. It would be further advantageous for the devices, systems, and methods to include sensors and electronic components to help determine how evenly and effectively a person is applying coverage and when a person should reapply coverage based on last use and application. Some embodiments of the present disclosure are also capable of capturing imagery to evaluate coverage, detect lesions, and/or measure external factors in the surrounding so as to provide feedback to the user. It would also be advantageous to have a means by which data collected from the device can be transmitted to third parties or reviewed by artificial intelligence (AI), or the like, so as to assist in the processes of anomaly detecting, monitoring, and/or diagnosing. In this way, a dermatologist, or the like, can review a user's data and prescribe a suitable therapy for the user. Additionally, a user can share collected data with friends or family members, so as to allow them to track and remind the user when application is needed.

### SUMMARY

Provided herein are example embodiments of systems, devices and methods for an applicator device for applying topical substances to the body, detecting anomalies, and collecting, processing and displaying data relating thereto. A skin protection, anomaly detection, and management system can be provided that includes an applicator device. In some embodiments, the applicator device is configured to collect data related to the skin or body and, through a communication interface, transmit the data to a remote device, application, or the like, whereby it is translated into a display output for the user (e.g., charts, tables, illustrations relating to a user's monitored skin data and trend information).

According to some embodiments, the applicator device can comprise a removable lid, a body, an actuator mechanism, a flexible tube, and a hollow arm having one or more nozzles. In exemplar embodiments, the body can comprise a base having a cover, and a receptacle configured to house a container comprising a fluid, such as a topical substance. The body can further comprise a tubing adaptor, wherein the tubing adaptor can include a valve inlet in airtight communication with a valve in the container. The valve inlet described herein is configured to receive the fluid, or topical substance (e.g., a gel, a cream, a serum, a skincare product, a makeup, a soap, a medicinal agent, a drug agent, a sunscreen, a tanning oil, a mosquito a repellant, a moisturizer, a lotion, and an aloe vera, etc.) from the container. In some embodiments, the tubing adaptor can include a central conduit and a valve outlet, wherein the topical substance received from the valve inlet is configured to pass through the central conduit and into the valve outlet.

In some embodiments, the applicator device can include one or more sensors. In exemplar embodiments, the one or more sensors can comprise one or more of an ultraviolet (UV) sensor, a pressure sensor, and a camera sensor. Furthermore, the applicator device can also include electronics disposed therein, wherein the electronics are operatively coupled to the one or more sensors and are configured to process signals received from the one or more sensors. The electronics can include one or more processors, a printed circuit board, a battery, a memory, a pump, communication circuitry (e.g., Bluetooth circuitry, Wi-Fi circuitry, etc.), and a universal serial bus (USB) connector.

The applicator device can further comprise a flexible tube comprising a channel, a tube inlet and a tube outlet. The tube inlet can be affixed to the valve outlet and is configured to receive the topical substance therefrom. Moreover, the channel is configured to receive topical substance from the tube inlet and deliver the topical substance to the tube outlet. In some embodiments, the flexible tube is configured to extend through the body in a proximal direction.

In some exemplar embodiments described herein, the applicator device includes a hollow arm configured to transition between a closed configuration and an extended configuration. The arm includes a first terminus and a second terminus, wherein the first terminus affixed to the removable lid, which is removably attached to the body. The hollow arms described herein also include a central channel extending from the first terminus to the second terminus, wherein the central channel is configured to receive at least a portion of the flexible tube. The arm further comprises one or more nozzles that are in fluid communication with the tube outlet and are configured to spray the topical substance therefrom. Further, when the hollow arm is in the extended configuration, the applicator device is able to expand the surface area that is covered by the spray dispersing from the one or more nozzles. Thus, the user is more conveniently able to reach and spray surface area on the user's body that could otherwise be missed or uncovered.

The applicator devices described herein can also include an actuator configured to cause the applicator device to spray the topical substance. Specifically, the actuator, upon being triggered by a force applied thereto, is configured to exert pressure onto the container. As a result, the pressure exerted on the container causes the topical substance to travel from the container into the one or more nozzles.

The embodiments provided herein are improvements to provide a device by which one can apply topical substances to hard-to-reach areas easily and in a manner so as to provide complete and adequate coverage. Other improvements and advantages are provided as well. The various configurations of these devices are described in detail by way of embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a side perspective view depicting an example embodiment of an applicator device with a hollow arm in the closed configuration.

FIG. 2 is a side perspective view of the applicator device depicted in FIG. 1, with the hollow arm in the extended configuration.

FIG. 3 is a system overview of an applicator device, remote device, local computer system, network, and remote system.

FIG. 4 is a block diagram depicting an example embodiment of the system depicted in FIG. 3.

FIG. 5 is a block diagram depicting an example embodiment of an applicator device in connection with multiple remote devices.

FIG. 6 is a block diagram depicting multiple applicator devices in connection with a single remote device.

FIG. 7 is a top perspective exploded view of the applicator device depicted in FIG. 1.

FIG. 8 is bottom perspective exploded view of the applicator device depicted in FIG. 1.

FIG. 9 is a close-up back perspective view depicting an example embodiment of an applicator device, wherein the removable lid is hingedly attached to the body.

FIG. 10 is a side close-up cross-sectional view depicting an example embodiment of an applicator device having electronics disposed in the removable lid.

FIG. 11 is a close-up cross-sectional view depicting an example embodiment of an applicator device having electronics disposed in the body.

FIG. 12 is a partially exploded side view depicting an example embodiment of an applicator device.

FIG. 13 is a side perspective view depicting an example embodiment of an applicator device.

FIG. 14 is a partially exploded bottom view depicting an example embodiment of an applicator device, illustrating the tubing adaptor.

FIGS. 15 through 17 are cutaway views depicting an example embodiment of an applicator device, illustrating the valve assembly, tubing system, and route by which fluid, such as a topical substance (or the like), travels through the device.

FIG. 18 is a side view depicting an example embodiment of an applicator device with a hollow arm having an appendage that interfaces with a posterior portion of the body.

FIG. 19 is a side perspective view depicting an example embodiment of an applicator device having an arm protruding from the body.

FIG. 20 is a top perspective view depicting one embodiment of an applicator device having a button, and a flexible tube disposed on an exterior of the device.

FIG. 21 is a top perspective view depicting one embodiment of an applicator device having a button, and a flexible tube disposed on an interior of the device.

FIG. 22 is a top perspective view depicting one embodiment of an applicator device having a button, and a flexible tube disposed on an interior of the device.

FIG. 23 is a top perspective view depicting one embodiment of an applicator device having a button, and a flexible tube disposed on an interior of the device.

FIG. 24 is a top perspective view depicting one embodiment of an applicator device having a button, and a flexible tube disposed on an interior of the device.

FIG. 25 is a side perspective view depicting an example embodiment of an applicator device having the hollow arm in the closed configuration.

FIG. 26 is a close-up perspective view depicting the applicator device of FIG. 25, illustrating the hollow arm in the extended configuration.

FIG. 27 is a perspective view of the applicator device depicted in FIG. 25, illustrating the hollow arm in the extended configuration.

FIG. 28 is a top perspective view depicting an example embodiment of an applicator device having the hollow arm in the closed configuration.

FIG. 29 is a perspective view of the applicator device depicted in FIG. 28, illustrating the hollow arm in an extended configuration.

FIG. 30 is a perspective view depicting an example embodiment of an applicator device having a telescoping assembly.

FIG. 31 is a perspective view of the applicator device depicted in FIG. 30, illustrating the inner telescoping base partially telescoped into the outer telescoping body.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

### General Overview of Applicator Device

Generally, embodiments of the present disclosure include systems, devices, and methods for applying a fluid, such as a topical substance to the body, detecting anomalies, and collecting, processing and displaying data related thereto. Specifically, an applicator device is described which provides a means for a user to apply the fluid or topical substance onto a user's body. Topical substances can include, but are not limited to, skin, body, and hair products. Specifically, topical substances can be an aerosol product or a non-spray product. More specifically, topical substances can include lotions, gels, creams, serums, skincare products, makeup, soaps, lotions, medicinal or drug agents, repellants, aloe vera, tanning oils, sunscreens, and the like. Even more specifically, the topical substances can present in oil-based, water-based, wax-based, or silicone-based form, and comprise a liquid, gas, or solid base.

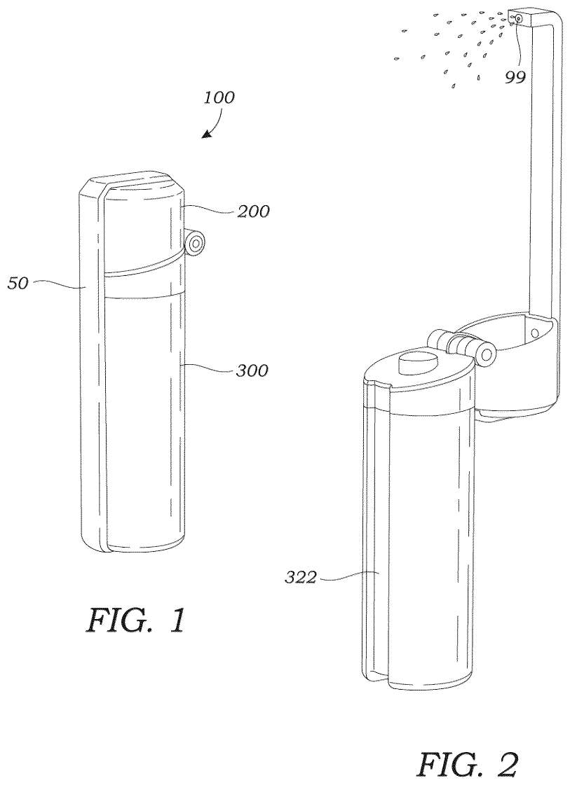

In FIGS. 1 and 2, an exemplar embodiment of an applicator device 100 is shown. The applicator device 100 comprises a body 300 configured to house the fluid or topical substance. The applicator device 100 is configured such that it can accommodate topical substances in cartridges, bottles, cups, vessels, containers, or the like, of varying shapes and sizes. Moreover, the applicator device 100 is configured such that an adaptor can be used which enables topical substances comprised in containers of non-standard sizes and dimensions to be utilized by the applicator device. Various topical substances, and sizes and dimensions can be determined by one skilled in the art.

As illustrated in FIGS. 1 and 2, applicator device 100 can comprise a removable lid 200 attached to a body 300, and a hollow arm 50 having one or more nozzles 99 residing or projecting from thereon. As will be further detailed below, the body 300 can be configured to house a fluid or topical substance. Further, the one or more nozzles 99 can be configured to spray the topical substances therefrom.

### Exemplary Embodiments of a Skin Protection, Anomaly Detection, and Data Management System

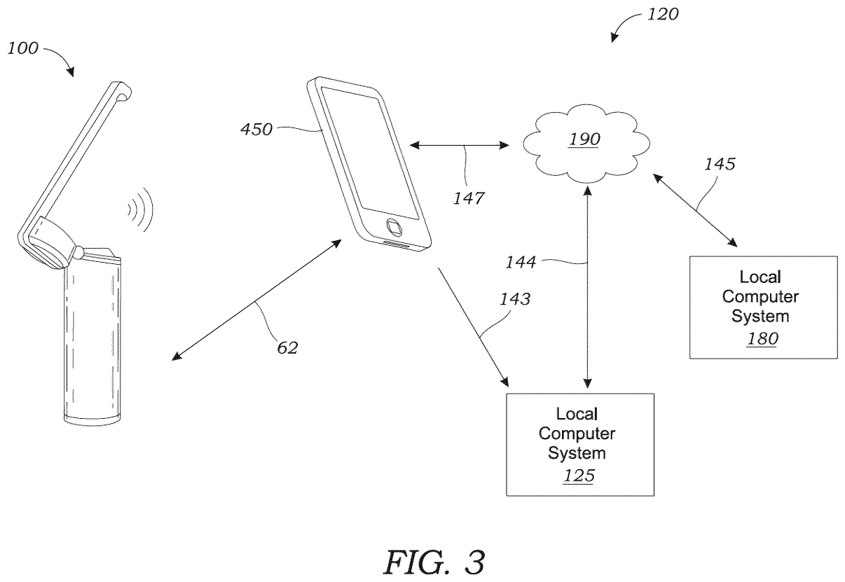

FIG. 3 is a conceptual diagram depicting an example embodiment of a skin protection, anomaly detection, and data management system 120 that includes an applicator device 100 and a remote device 450. Applicator device 100 is further described below and can communicate with remote device 450 via a communication path 62 using a wired or wireless technique. Example wireless protocols include Bluetooth, Bluetooth Low Energy (BLE, BTLE, Bluetooth SMART, etc.), and others. Remote device 450 can communicate with local computer system 125 via a communication path 143 using a wired or wireless technique. Local computer system 125 can include one or more of a laptop, desktop, tablet, phablet, smartphone, wearable device (e.g., smart watch), set-top box, or other computing device and wireless communication can include any of a number of applicable wireless networking protocols including Bluetooth, BLE, Wi-Fi or via a communications path 144 with network 190, by wired or wireless technique as described previously, and can communication similar to how remote device 450 can communicate via a communications path 147 with network 190. Network 190 can be any of a number of networks, such as private networks and public networks, local area or wide area networks, and so forth. A trusted computer system 180 can include a server and can provide authentication services and secured data storage and can communicate via communications path 145 with network 190 by wired or wireless technique.

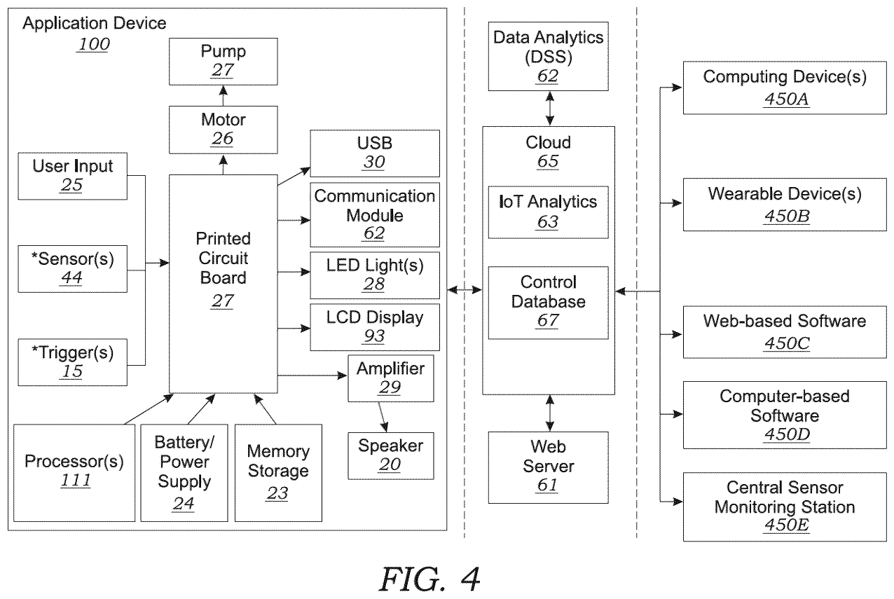

FIG. 4 is a block diagram depicting an example embodiment of an applicator device 100 configured with electronics that can have the majority of the processing capability for rendering end-result data suitable for display to the user. Here, applicator device 100 can include a user input 25 component, trigger(s) or actuator(s) 15, a printed circuit board (PCB) 22, and motor 26 and a pump 27 operatively coupled with a processor 111. Processor 111 can include one or more processors, microprocessors, controllers, and/or microcontrollers, each of which can be a discrete chip or distributed amongst (and a portion of) a number of different chips. Also included can be memory 23 and power supply 24. Memory 23 can be volatile and/or non-volatile memory. The power source 24 can be a coin cell battery, mini-solar panel, micro-solar panel, or the like. As understood by one of skill in the art, these components are electrically and communicatively coupled in a manner to make a functional device. Additional features can also be included in the applicator device 100, including but not limited to: a USB connector or port 30; a communication module 62 (e.g., Bluetooth, BLE, Wi-Fi, etc.); light emitting diode (LED) light 28 configured to activate as a visual signal to find the applicator device 100; LED light(s) 28 configured to indicate remaining battery, remaining fluid in applicator device 100, or faults and errors detected in device 100; a Liquid Crystal Display (LCD) display 93; an amplifier 29; and a speaker 20.

The applicator device 100 can also include one or more sensors 44 which can be operatively coupled with the processor 111. In this way, the processor 111 can collect information and data from the sensor(s) 44, and transfer said data directly through a wired connection (USB) or via a wireless connection (e.g., Bluetooth, BLE) to a connected device 450 (smart phone, watch, tablet, etc.), firmware, software, or the like. The device 100 can comprise the following sensor(s) 44: a UV sensor configured to detect electromagnetic radiation of UV spectrum with wavelength of 10 nm to 400 nm; a visible light sensor or photodiode configured to detect light wavelengths of the visible light spectrum and can be used as mini solar cell to power the device 100; an infrared (IR) sensor configured to detect electromagnetic radiation wavelengths of infrared radiation; a temperature sensor configured to determine outside temperature near the device 100, or temperature inside the device 100; a gyroscope sensor configured measure rotational changes of the device 100; an accelerometer configured to measure changes in acceleration of a device 100 to detect position, velocity, vibration, and orientation; an ultrasonic sensor configured to detect proximity of device 100 to objects; a flex sensor configured to measure the flex of a device 100 (e.g. for a flexible arm 50 on the device 100); a tilt sensor configured to measure the degree of tilt of a device 100; a touch sensor configured to identify that the device 100 is being touched, held or in use; a humidity sensor configured to sense the humidity external to the device 100; a camera sensor configured to capture pictures or videos of various resolution, frames per second (for burst photos or videos), and Field of View (FOV); a flow sensor configured to measure aerosol or lotion flow through tubing system 730; a Global Positioning System (GPS) sensor configured to determine location of device 100; an alcohol sensor configured to detect the presence of alcohol; a pressure sensor configured to measure the amount of pressure applied to buttons or actuators 15 on device 100; and, a force sensor configured to measure amount of force applied to actuators 15 on the device 100.

In some embodiments, the electronics are configured to process raw data and signals received from the one or more sensors 44. In the exemplar embodiment depicted in FIG. 4, the applicator device 100 is in communication with an analytics and data storage unit, to which processed data is transmitted. The analytics and data storage unit can include a cloud 65 comprising Internet of Things (IoT) analytics 63 functionality and a control database 67. In some embodiments, the cloud 65 is configured to organize, filter and sort the received data. The cloud 65 can also be in communication with a web server 61 and a data analytic unit 62. In some embodiments, the cloud 65 is configured to output the data to the web server 61 and data analytics unit 62, which in turn processes the received data to arrive at the end-result of skin data and trend values, etc. This data can then be provided to communication circuitry for sending to a connected device 450, for example, where minimal further processing is needed by the resident software application to display the data. In some embodiments, the connected device 450 can include any of the remote devices 450 mentioned above, a computing device 450A, a wearable device 450B, web-based software 450C, computer-based software and/or device 450D, a central sensor monitoring station 450E, or the like. The connected device 450 can also interface, via internet, for example, with a public data source or private data source, or create a data connection with a mobile application (e.g., health applications related to or for an Android Operating System, Bada, Blackberry Operating System, Windows Mobile Operating System, Symbian Operating System, Palm Operating System, Web Operating System, or iPhone Operating System, etc.).

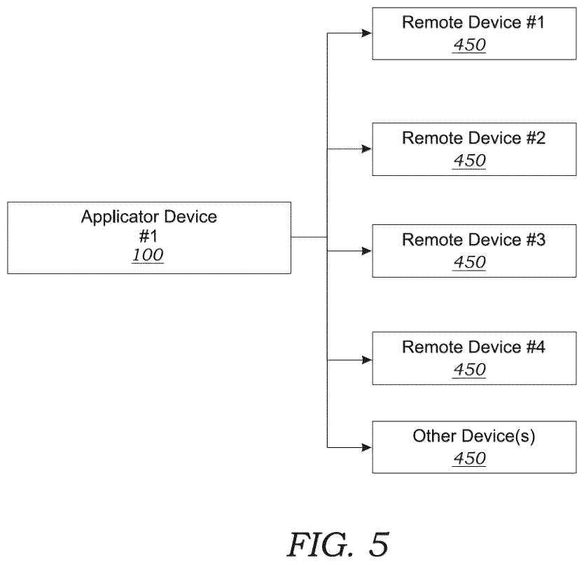

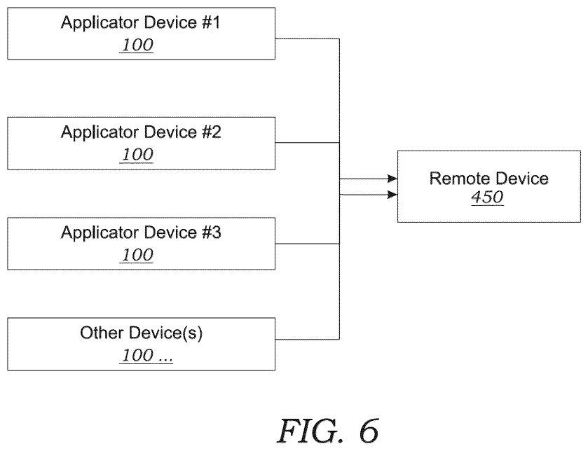

As illustrated in FIG. 5, data from one applicator device 100 can be shared with one or more computing or remote devices 450. In this way, a single user can give grant third parties permission to access data obtained from the applicator device 100. As shown in FIG. 6, a plurality of applicator devices 100 can transmit and share data to one software application. For example, a single user can connect and monitor multiple applicator devices 100 in one software application on their remote device 450 (e.g., phone, watch, tablet) or web-based or computer-based software application. In this way, the user can request and review data from multiple users.

### Example Embodiments of Applicator Devices

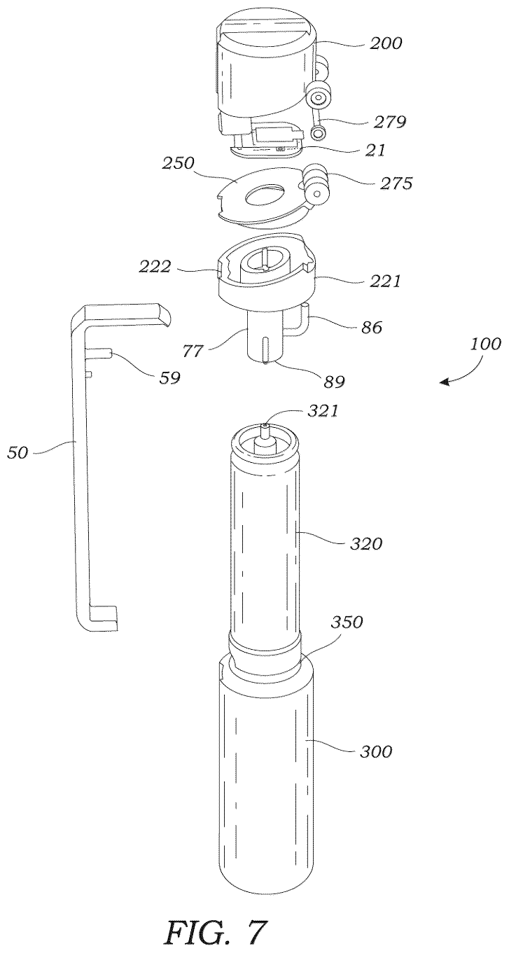

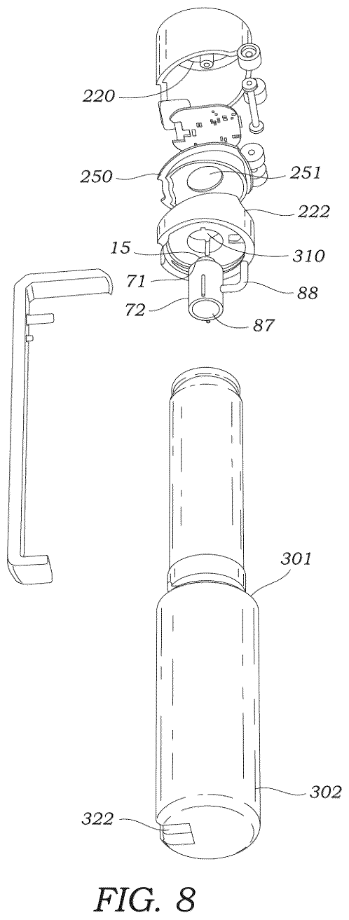

FIGS. 7 and 8 are exploded views of one example embodiment of applicator device 100. According to an aspect of the depicted embodiments, applicator device 100 can comprise: a hollow arm 50, a body 300 having a first end 301 and a second end 302; and a removable lid 200. In some embodiments, the applicator device 100 can further comprise one or more sensors 44 (not shown) and/or electronics 21.

In exemplar embodiments, the hollow arm 50 is extendable, rotatable, and/or bendable. In some embodiments, the hollow arm can have an elongate structure. The hollow arm 50 functions so as to enhance the device's 100 reach and, thus, cover hard-to-reach spots on a user's body. In some embodiments, the arm 50 is replaceable. In some embodiments, the arm 50 is removable. For example, the arm 50 can be unscrewed and screwed in certain embodiments. Furthermore, the hollow arm 50 is configured to move along a longitudinal axis, so as to comprise a closed configuration and an extended configuration. In the closed configuration, the arm 50 is configured to interface with at least a portion of the body 300. For example, the body 300 can comprise a recess 322 (best shown in FIG. 2) sized and configured for receipt of at least a portion of the arm. In this manner, the arm 50 can interface with the body 300. In some embodiments, the arm 50 interfaces with the recess 322 (not shown) through press-fit engagement. Moreover, in some exemplar embodiments, the hollow arm 50 bevels into the recess 322 so as to not protrude or substantially protrude therefrom. In this way, an exterior surface of the arm 50 and an exterior surface of the body 300 are aligned along the same longitudinal axis.

In some embodiments, the recess longitudinally extends from the first end 301 to the second end 302. In some embodiments, the recess 322 can extend from the first end 301 to a posterior of the body 300. In the extended configuration, the arm 50 is not engaged with the recess 322. In some embodiments, the arm 50 extends up to at least a 1800 angle relative to the longitudinal axis of the body 300.

The body 300 includes a receptacle 350, a base 221, and a cover 250. In some embodiments, the base 221 includes a housing 222. In some embodiments, the body 300 and receptacle 350 are tubular or cylindrical in shape. Various body and receptacle 350 sizes, dimensions, shapes, configurations, and constructions can be determined by those skilled in the art. The receptacle 350 is configured such that the container 320 can be loaded and housed therein. The receptacle 350 also functions as a connector to hold the container 320 in a tight position such that it is stable when connected to the body 300. In some embodiments, the container 320 containing a fluid, such as a topical substance (or the like) is an aerosol spray can.

Referring still to FIGS. 7 and 8, the cover 250 of the applicator device 100 is positioned directly adjacent and proximal to the base 221, and is sized and configured so as to overlay on the base 221. In some embodiments, the cover 250 encases the housing 222 or an upper portion of the base 221. In this way, the cover 250 can serve as a protective mechanism to cover the one or more sensors 44 and/or electronic(s) 21 that can reside distally therefrom and in the body 300. Further, as shown in in FIGS. 7-8, the cover 250 comprises a cavity 251, wherein a lever, a switch, a button, an actuator 15, or the like, can protrude proximally or upwardly through, and extend therefrom. In this way, the cover 250 functions as a housing for the actuator 15, or the like. In some embodiments, the cavity 251 forms a ring and is substantially annular in shape. In one example embodiment, the cavity 251 resides along a central axis of the cover 250. In some embodiments, the cover 250 and the body 300 reside along the same central axis.

In some embodiments, the base 221 further includes an opening 310 sized and configured for receipt of a tubing adaptor 77. In some embodiments, this opening 310 forms a ring and is substantially annular in shape. Further, the opening 310 of the base 221 and the cavity 251 of the cover 250 are sized and configured so as to be complementary to one another. In one example embodiment, the opening 310 resides along a central axis of the base 221. In some embodiments, the central axis of the base 221 and the central axis of the body 300 are the same. In one example embodiment, an inner circumference of the base 221 housing defines an outer circumference of the base opening 310, and an outer circumference of the base housing 222 defines an exterior of the base 221.

According to another aspect of the embodiments, the tubing adaptor 77 includes a valve assembly 88. The valve assembly 88 comprises a valve inlet 89, a central conduit 87, and a valve outlet 86. In some embodiments, the tubing adaptor 77 includes the actuator 15, or the like (which will be detailed further below). In at least one embodiment, the actuator 15 is integrated into the tubing adaptor 77 and is configured to extend from the cover 250 so as to be depressible by a user when the device's lid 200 is disengaged from the body 300. In one example embodiment, the tubing adaptor 77 comprises a proximal portion 71 and a distal portion 72, wherein the actuator 15 is configured to extend from the proximal portion 71, and wherein the valve inlet 89 extends from the distal portion 72. In some embodiments, wherein the opening 310 and the cavity 251 are complementary in size and shape, the button 15 can extend in a proximal direction through the opening 310 and cavity 251 so as to protrude from the cavity 251. In this way, the tubing adaptor 77 acts as an activator that provides downward force on the container 320 when the button 15 is pressed, thereby causing the topical substance to spray up into tubing system 850.

Moreover, the valve inlet 89 is configured to attach to a portion of the container 320 so as to receive the topical substance disposed in the container 320. In some examples, as illustrated in FIGS. 7-8, the container 320 comprises an applicator cap that is removable, and wherein once removed, exposes a valve 321 on the container 320 that functions as a passageway to the topical substance disposed therein. In some example embodiments, the valve inlet 89 is configured to engage with the valve 321 and create tight seal. In this way, the valve inlet 89 is in airtight communication with the valve 321 and receives topical substance therefrom. In some embodiments, the tubing adaptor 77 is composed of silicone, rubber, or material of the like, so as to provide the tight seal around the valve inlet 89 and valve 321.

The removable lid 200 is configured to be mateable with the body 300. In this way, the removable lid 200 can engage and disengage with a portion of the body 300 so as to provide an open configuration and closed configuration for the applicator device 100. When engaged with a portion of the body 300, the removable lid 200 includes a bottom portion 220 which interfaces with at least a portion of the cover 250. In this configuration, the cover 250 is enclosed by the removable lid 200. When disengaged from the body 300, the cover 250 is exposed and no longer interfacing with the removable lid 200. In some embodiments, the removable lid 200 is threadably engaged with the body 300. Specifically, the removable lid 200 comprises female threads which affix to male threads of the body 300. In some embodiments, the lid 200 is twistable and is removed by way of the user twisting the removable lid 200 off the body 300.

In some embodiments, the removable lid 200 is hingedly attached to the body 300. In exemplar embodiments, the removable lid 200 is hingedly attached to the body 300 by a hinge 279 and a shoulder portion 275. FIG. 7-8 depicts an example embodiment where the cover 250 further comprises the shoulder portion 275, which engages with a hinge 279 so as to connect the body 300 with the removable lid 200. In the illustrated embodiment, the hinge 279 attaches to a central point on the shoulder portion 275 so as to allow pivotal opening and closing along the central point. In this manner, the cover 250 further serves as a base for the hinge 279 to connect to the body 300 thereby.

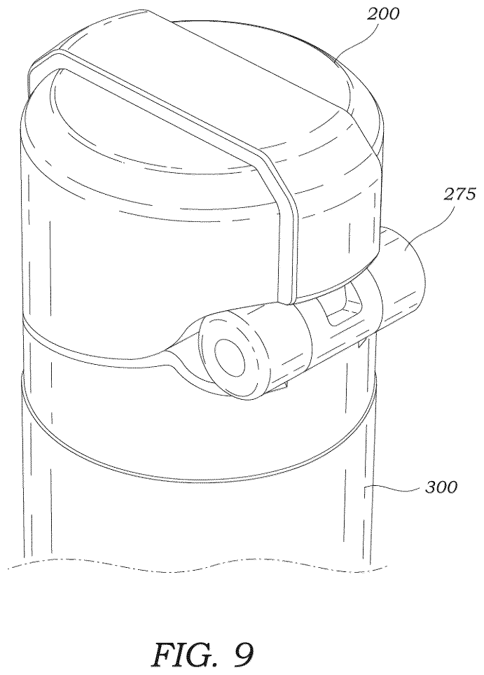

FIG. 9 is a close-up back perspective view of the applicator device depicted in FIGS. 7 and 8, illustrating the removable lid 200 hingedly attached to the body 300 by the shoulder portion 275. Further, in some embodiments, electrical wiring (not shown) can run through the shoulder portion 275.

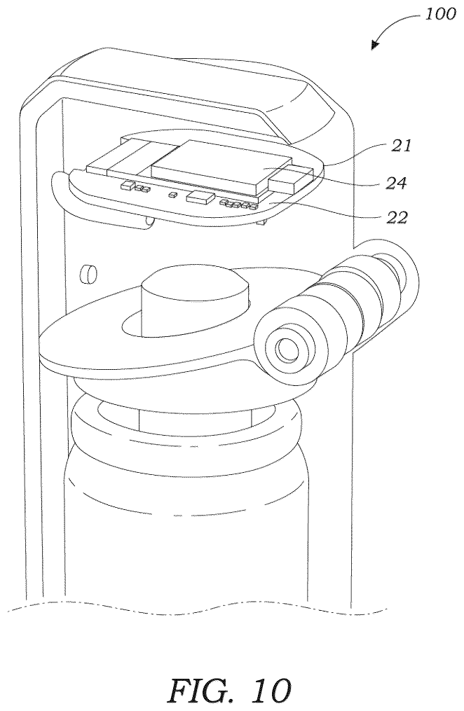

FIG. 10 is a side close-up cross-sectional view depicting some components of an example embodiment of the applicator device 100. As shown in FIG. 10, some embodiments of the applicator device 100 can include electronics 21 (e.g., PCB 22, battery 24, etc.) can be disposed in the lid 200. In some embodiments, one or more sensors 44 (not shown) can be disposed in the lid 200. In certain embodiments, electronic components 21 can be disposed in an interior portion of the lid 200. FIG. 10 illustrates an example embodiment of the applicator device 100, wherein the battery 24 and PCB 22 are disposed within the removable lid 200.

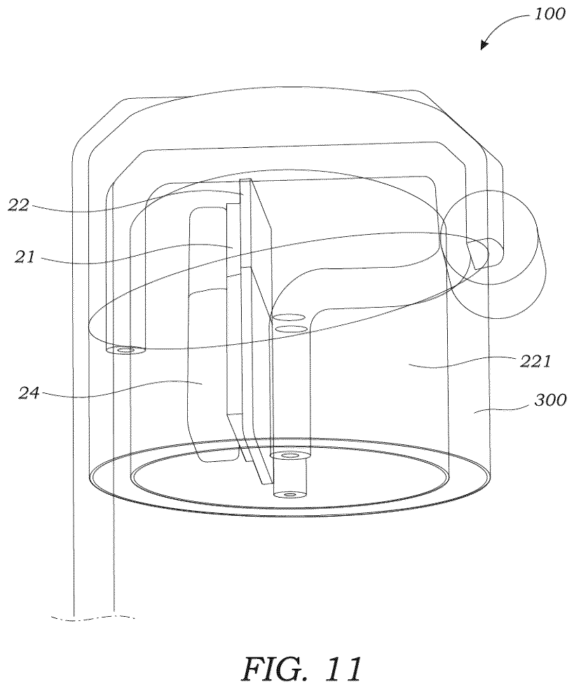

FIG. 11 is a close-up cross-sectional view depicting some components of an example embodiment of the applicator device 100. As shown in FIG. 11, electronics 21 can be disposed in the body 300. For example, electronics 21, such as the PCB 22 and battery 24 can be disposed in the base 221. In some embodiments, the electronics 21 can be disposed in the housing 222.

In some embodiments, one or more sensors 44 can be disposed in the body 300. Specifically, according to some embodiments, the one or more sensors 44 can be disposed in the base 221. More specifically, in some embodiments, the one or more sensors can be disposed in the housing 222. In one example embodiment, the one or more sensors 44 are a force sensor and/or pressure sensor. Additionally, in some embodiments, the one or more sensors 44 can include a UV sensor and/or a camera sensor. Other sensors 44, as detailed above or as determined by those skilled in the art may also be included in the housing.

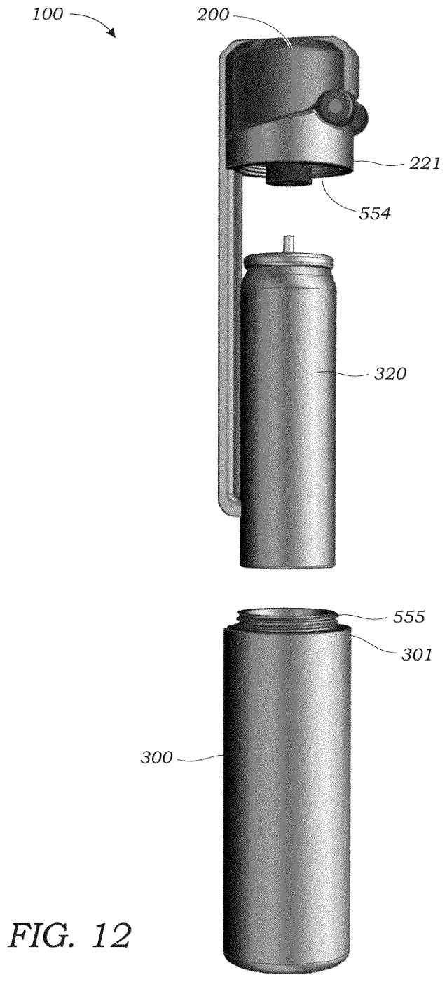

FIG. 12 is a partially exploded side view depicting an example embodiment of the applicator device 100. As shown in FIG. 12, the container 320 can be loaded from the first end 301. In these embodiments, male threads 555 on the first end of the body 300 threadably affix to female threads 554 on an underside of the base 211. In this way, the user can screw and unscrew the removable lid 200 portion from the body 300 so as to load the device 100 with a desired container 320.

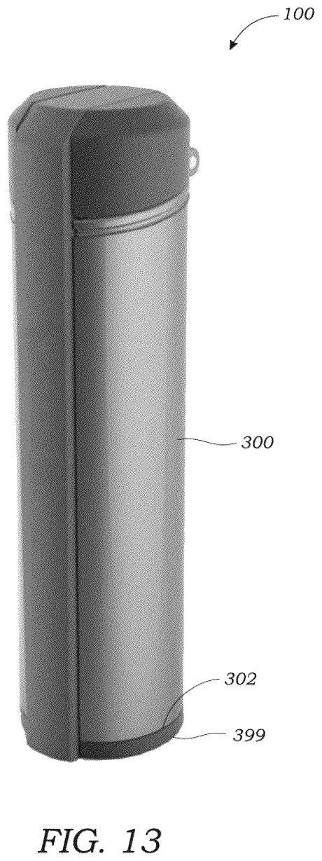

FIG. 13 is a side perspective view depicting an example embodiment of the applicator device 100. As illustrated in FIG. 13, the body 300 can include a removable cap 399. Specifically, in some embodiments, the second end 302 can comprises the removable cap 399. The removable cap 399 can mate with the body's 300 second end 302 through a press-fit engagement. In some embodiments, the removable cap 399 can be screwed and unscrewed. In some embodiments, the cap 399 can include female threads so as to threadably affix with male threads on the second end 302 of the body 300. In other embodiments, a button or latch mechanism can be utilized to assist in process of engagement and removal of the removable cap 399. In yet other embodiments, the removable cap 399 can engage with the body 300 by a hinge mechanism. When the removable cap 399 is removed from the body 300, the receptacle 350 (not shown) is accessible to a user. Thus, when the removable cap 399 is disengaged form the body 300, a user can insert the container 320 (not shown) into the receptacle 350 or, remove the container 320 (not shown) therefrom.

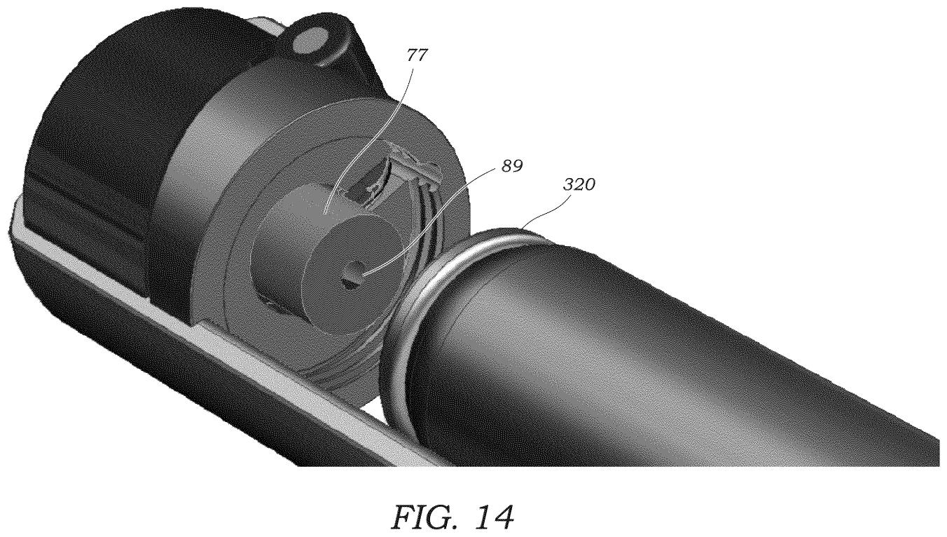

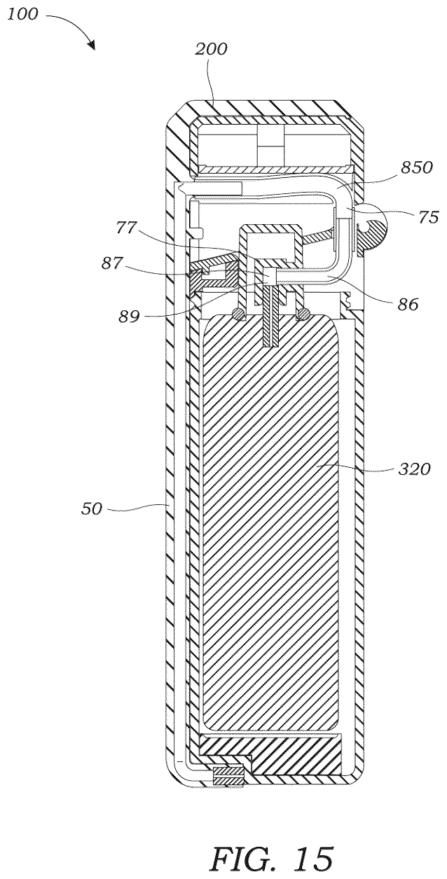

FIG. 14 is a partially exploded bottom view depicting the tubing adaptor 77 of the applicator device 100. As shown in FIG. 14, the valve inlet 89 is configured to attach to a portion of the container 320 so as to receive the topical substance from the container 320.

FIG. 15 is a cross-sectional view depicting a tubing system 850 of an example embodiment of the applicator device 100. Specifically, the tubing system 850 comprises the valve assembly 88 and the flexible tube 75. In FIG. 15, the applicator device 100, the hollow arm 50 is in a closed configuration. As shown in FIG. 15, the central conduit 87 of the tubing adaptor 77 is configured such that fluid or topical substance expelling from the container 320 can pass through the valve inlet 89 and through the valve outlet 86. The valve outlet 86 is configured to be in fluid communication with the flexible tube 75. In some embodiments, the valve outlet 86 extends from a left or right side of the tube adaptor 77. In these embodiments, the tube adaptor 77 comprises a C-shaped, U-shaped or V-shaped valve outlet 86, wherein one end of the valve outlet 86 extends from the tube adaptor 77 and the other end of the valve outlet extends in a proximal direction towards the lid 200. In this way, the valve outlet 86 guides the flexible tube 75 connected thereto in the proximal direction.

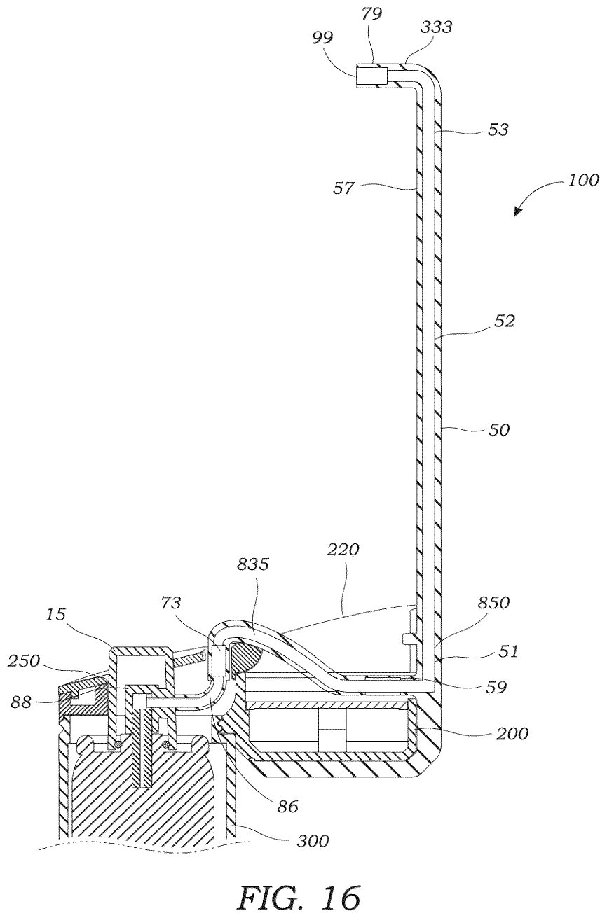

FIG. 16 is a cutaway view depicting an example embodiment of a tubing system 850 of the applicator device 100. Specifically, FIG. 16 depicts the applicator device 100 of FIG. 15 with the hollow arm 50 in an extended configuration. As shown in FIG. 16, the flexible tube 75 includes a channel 835, a tube inlet 73 and a tube outlet 79. The flexible tube 75 extends from the tube inlet 73, which is interfacing with the valve outlet 86, and ends at the tube outlet 79, which interfaces with one or more nozzles 99 residing or projecting from the hollow arm 50.

More specifically, the valve assembly 88 is in fluid communication with the flexible tube 75 by way of the valve outlet 86 being affixed to the tube inlet 73. In this manner, the tube inlet 73 is configured to receive fluid or topical substance traveling through the valve outlet 86. And even more specifically, the topical substance received at the tube inlet 73 can pass through the channel 835 such that it ultimately expels out of the tube outlet 79. Moreover, the tube outlet 79 is in fluid communication with one or more nozzles 99 on the hollow arm 50. Thus, the fluid or topical substance can exit the tube outlet 79 and disperse out of the one or more nozzles 99 on the hollow arm 50.

To actuate the applicator device 100 so as to disperse a spray through the one or more nozzles 99, the actuator or button 15 must be triggered. A manual or automatic force can be used to trigger the actuator 15. For example, user can depress a button 15 down in order to initiate actuation. Upon the actuator 15 being triggered and actuation initiated, pressure is exerted onto the container 320. For example, a button 15 protruding from the cover 250 that is then pushed down will exert downward pressure onto the container 320, thereby causing the fluid or topical substance to exit out of the container 320 and into the valve assembly 88. In some embodiments, the device 100 leverages the already-existing pressure in the container 320 to mechanically deploy topical substance out of the device 100 when the actuator 15 is triggered.

In some embodiments, a small electric pump exists inside the device 100 and serves as a mechanism for driving topical substance out of the container and through the tubing system 850, so as to spray out of the one or more nozzles 99 of the device 100. In some embodiments, electric pump can continuously trigger actuation, thereby causing the topical substance to be continuously sprayed out of the container 320, into the tubing system 850, and out the nozzle(s) 99. In some of these embodiments, the mechanism comprises an on/off switch so as to allow continuous actuation when turned on. In other embodiments, a user can trigger actuation on the device 100 (e.g., pushing or squeezing a button 15), wherein one trigger equates to one discrete spray. In other embodiments, one trigger can equate to a continuous spray.

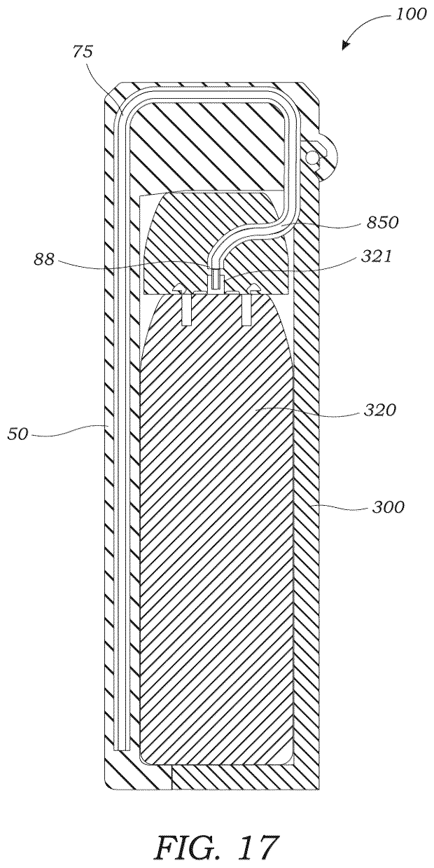

Moreover, the hollow arm 50 includes a first terminus 51, a second terminus 53, and a central channel 52. The central channel 52 expands from the first terminus 51 to the second terminus 53. In exemplar embodiments, the first terminus 51 affixes to or is integrated with the removable lid 200. As illustrated in FIGS. 17, in some embodiments, the arm 50 comprises a connector 59 (best shown in FIGS. 7 and 8), near or at the first terminus 51, which extends through a slot in the lid 200. The connector 59 functions to secure the arm 50 to the lid 200. Further, the connector 59 functions as a means for the tube 75 to connect with the arm 50. Specifically, the connector 59 comprises an aperture through which the tube 75 enters the central channel 52. In some embodiments, the tube 75, travels across the removable lid 200 (externally across the bottom portion 220, or internally) and enters the central channel 52 through the connector 59, so as to extend into the hollow arm 50. In this way, the lid 200 serves as a guide for the tube 75 as it routes the tube 75 into the arm 50.

The second terminus 53 of the arm 50 can comprise an appendage 333. In exemplar embodiments, the one or more nozzles 99 reside on or project from the appendage 333. In some embodiments, one or more nozzles 99 can project or reside along a longitudinal axis of the arm 50 (not shown). Specifically, in some embodiments, the one or more nozzles 99 can reside along an interior surface 57 of the arm 50. In yet other embodiments, nozzles 99 can reside on or project from both the appendage 333 and along the longitudinal axis of the arm 50. The one or more nozzles 99 are connected to the spraying source, or the topical substance, via the tubing system 850, and produce small droplets of the topical substance forming a dispersion of mist. In this way, when the hollow arm 50 is in the extended configuration, the applicator device 100 is able to expand the surface area that is covered by the spray dispersing from the one or more nozzles 99. Thus, the user is more conveniently able to reach hard-to-reach body areas in a manner so as to provide complete and adequate coverage of topical substance onto desired body parts.

The nozzle(s) 99 can use different spray pattern orifice cups or an adjustable orifice, the spray can be tailored to a user's requirements, allowing a small cone, medium cone, or large cone of spray. The device 100 can incorporate more complex constructions for providing selectable and controllable spray patterns and settings. Additionally, in some embodiments, the nozzle(s) 99 can incorporate different spray patterns such as a fine spray, a coarse spray, a jet spray, a wide spray, or a pulsating spray. Other spray patterns, sizes, settings, and nozzle configurations can be determined by those skilled in the art.

In some embodiments, the arm 50 can comprise primary and ancillary nozzles so as to expand the surface area that is covered by the spray released from the nozzles 99. The device 100 can further comprise two spray modes. Specifically, in some embodiments, the device 100 can comprise a first set of nozzle(s) 99 on the arm 50, and a second set of nozzle(s) 99 on the lid 200. For example, a second nozzle set 95 can project from or reside on the lid 200, proximally from the shoulder portion 275. In this embodiment, when the lid 200 is engaged with the body 300, and the device 100 is in a “closed” position, the device 100 utilizes a mechanism (e.g., a ball joint) to guide the topical substance into the second set of nozzle(s) 99. In this manner, the spray is released from the lid nozzle(s) 99, and not the arm nozzle(s) 99. In this way, the user can be able to utilize the device to spray body parts that can not require the reach of an extended arm 50. In an example embodiment, the user can apply downward pressure onto the lid 200 so as to actuate the mechanism and release spray from the lid nozzle(s) 99. Conversely, in this embodiment, when the lid 200 is disengaged with the body 300, and the device 100 is in a “open” position, the device 100 guides the topical substance into the first set of nozzle(s) 99. In this manner, the spray is released from the arm nozzle(s) 99, and not the lid nozzle(s) 99. In some embodiments, a diverter can be utilized so as to selectively permit or obstruct the flow of topical substance into outlets of each respective set of nozzles 99. Those skilled in the art will appreciate that other types of mechanisms can be utilized to vary spray modes in any of the embodiments described herein.

FIG. 17 is a cutaway view of the applicator device 100 depicted in FIGS. 15 and 16. FIG. 17 illustrates a route (shown by solid lines) by which topical substance or the like travels from the container and through the applicator device 100. Specifically, FIG. 17 depicts the topical substance's route from the valve 321, and through the tubing system 850. More specifically, FIG. 17 illustrates the topical substance's route from the valve assembly 88 of the tubing system 88, and through the flexible tube 75 which extends from the body 300 and into the hollow arm 50.

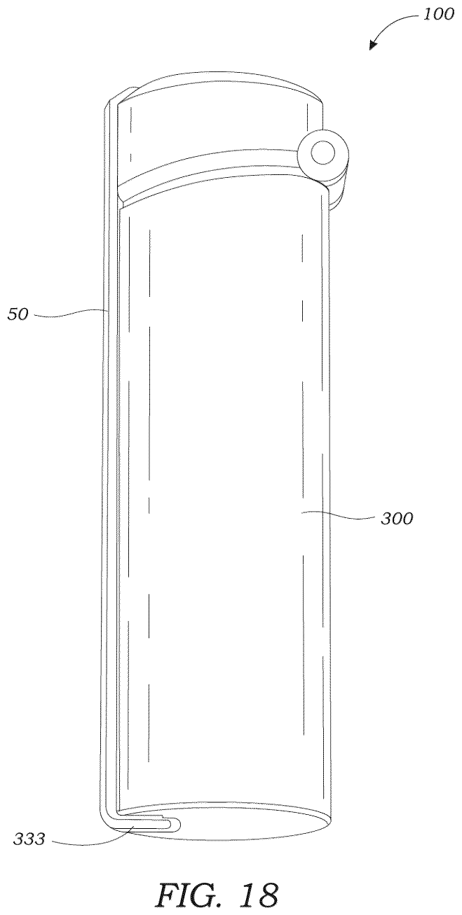

FIG. 18 is a side view depicting an example embodiment of the applicator device 100, wherein the appendage 333 interfaces with the body 300 when the applicator device 100 is in the closed configuration. In some embodiments, as shown in FIG. 18, the arm 50 can extend to a posterior portion of the body 300 so as to interface therewith and house the appendage 333 underneath the body 300 when in the closed configuration. In some embodiments, the body is configured such that the appendage 333 can be housed on an exterior of the body without protruding or substantially protruding therefrom.

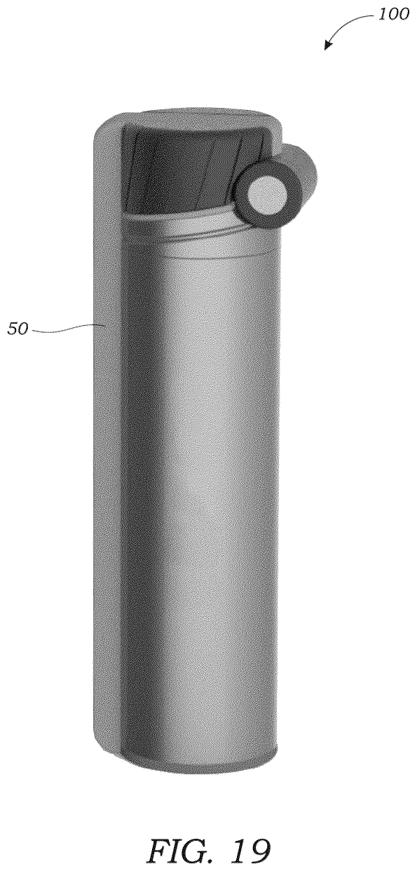

FIG. 19 is a side perspective view depicting an example embodiment of an applicator device 100 having the hollow arm 50 protruding from the body. As shown in FIG. 19, the arm 50 interfaces with the recess 322 (not shown) such that it protrudes therefrom. In this way, the longitudinal axis of the hollow arm's 50 exterior surface protrudes farther than the longitudinal axis of the body's 300 exterior surface.

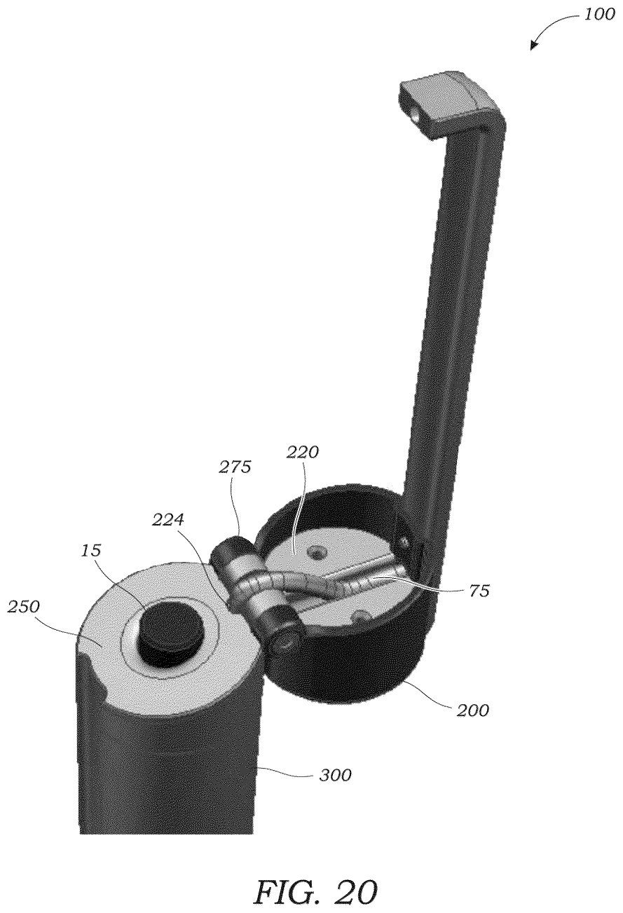

FIG. 20 is a top perspective view depicting one embodiment of an applicator device 100 having a button 15, and a flexible tube 75 disposed on an exterior of the applicator device 100. In some embodiments, the flexible tube 75 is configured to extend proximally through the body 300, passing through a space 224 in the cover 250. As illustrated in FIG. 18, in some embodiments, the tube 75 interfaces with the shoulder portion 275 as it extends in the proximal direction towards the removable lid 200. Further, the tube 75 can also interface with the bottom portion 220 of the lid 200. In this way, the tube 75 runs exterior to the lid 200. In one example embodiment, both the tube 75 and electrical wiring (not shown) interface with the shoulder portion 275.

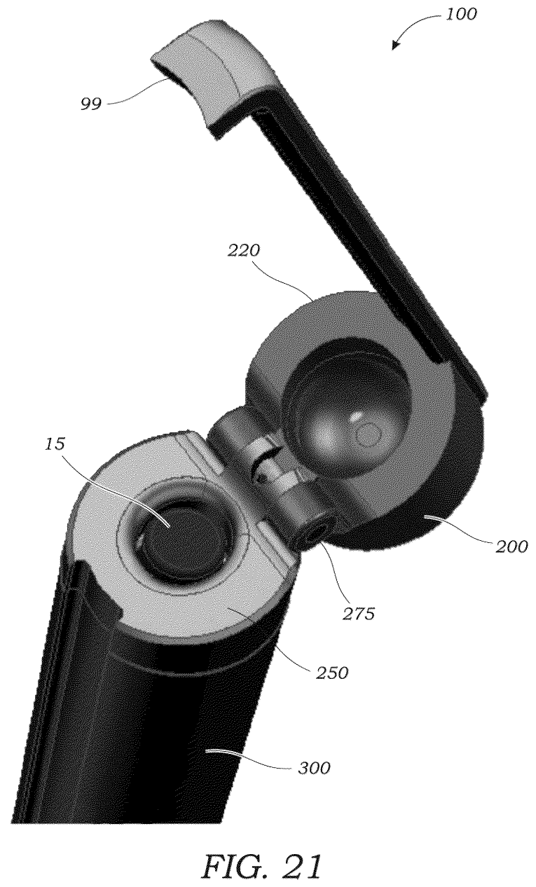

FIG. 21 is top perspective view depicting an example embodiment of an applicator device 100 having a button 15, and a flexible tube 75 (not shown) disposed on an interior of the applicator device 100. In some embodiments, the tube 75 (not shown) runs internal to the entire applicator device 100. In some embodiments, the bottom portion 220 comprises a concave midpoint, wherein the flexible tube 75 (not shown) travels along the outer circumference of the concave midpoint.

In some embodiments, the flexible tube 75 (not shown) is guided within a chamber of shoulder portion 275. For example, the flexible tube 75 (not shown) can extend through an internal section of the central point of the shoulder portion 275. In this manner, the flexible tube 75 (not shown) runs interior to the lid 200. In some embodiments, the flexible tube 75 (not shown) is routed along the central point of the shoulder portion 275 and a central axis of the lid 200. In exemplar embodiments, the central point and the central axis are aligned along the same axis.

The actuator mechanism 15, which can comprise a button, switch, toggle, tab, lever, or other actuator of the like or determined by one skilled in the art, can reside on the lid 200, or body 300 of the device 100. According to some embodiments, the button 15 extends from the side of the body 300 or the side of the lid 200. In some embodiments, the button 15 extends from a groove on the side of the body 300 or lid 200 and can be pushed or slid downwards or inwards so as to initiate actuation. In this way, the user is able to hold the device 100 in a closer proximity to the user's body, thereby maximizing the nozzle's 99 reach and spray surface area covered on the user's body.

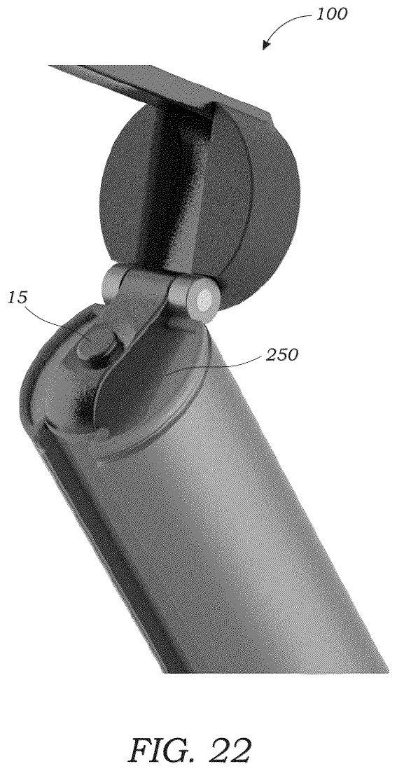

In exemplar embodiments, and as shown in FIG. 21, the button 15 is disposed along the central axis of the cover 250 and/or body 300. FIGS. 22 through 24 are top perspective views of various example embodiments of an applicator device 100 having a button 15 disposed along the central axis of the cover 250 and/or body 300. In other embodiments, the button 15 can extend from the cover 250 but in an off-center fashion. For example, the button 15 can reside near an outer edge of the cover 250 so as to be more reachable by a user's fingers, thereby improving ergonomics.

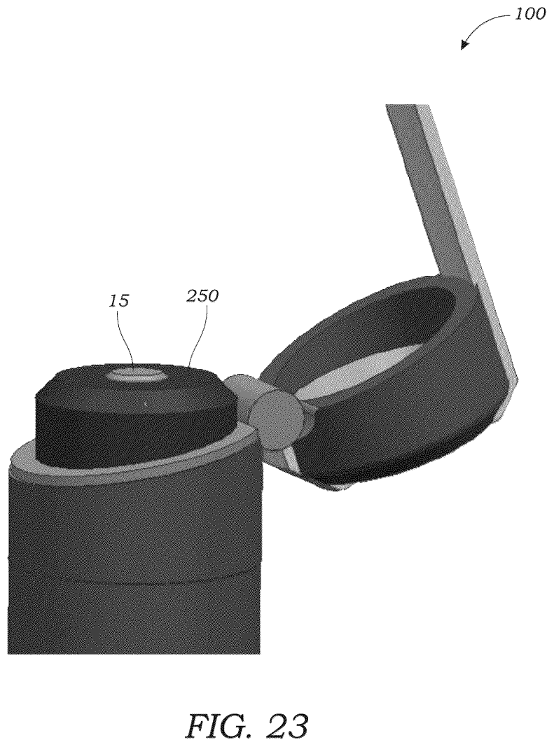

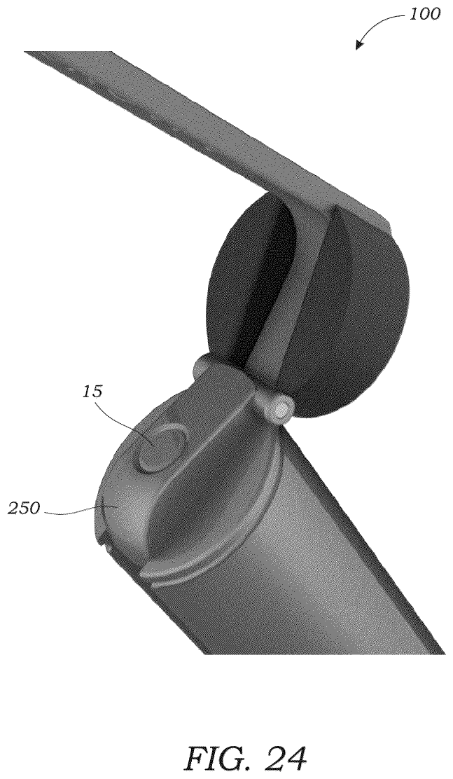

As shown in FIGS. 20 and 21, some exemplar embodiments of the applicator device 100 include a button 15 protruding from at least a portion of the cover 250. In some embodiments, as shown in the embodiments depicted in FIGS. 23 and 24, a tip of the button 15, when not depressed, is planar to a surface of the cover 250. In some embodiments, as shown in FIGS. 22 through 24, a portion of the cover's 250 surface is raised so as to form a hump. In this embodiment, the button 15 is caved on the hump or raised portion, and the tube 75 (not shown) can travel underside the raised portion.

In some embodiments, the applicator device 100 includes a locking mechanism to prevent the chance of inadvertent actuation. The locking mechanism can comprise a lock, a tab, or the like. In these embodiments, the locking mechanism has a first configuration and a second configuration. In the first configuration, the locking mechanism is configured to impeded accidental firing or triggering of the actuator 15. In the second configuration, the locking mechanism is configured to enable triggering of the actuator 15.

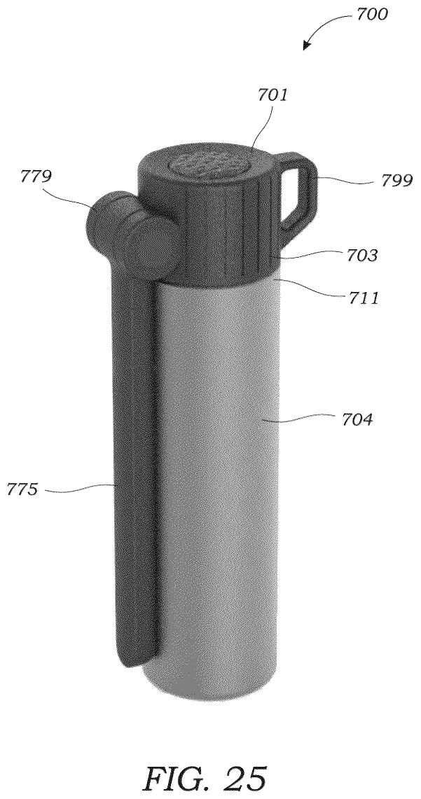

FIGS. 25 through 27 depict an example embodiment of an applicator device 700. As shown in FIG. 25, applicator device 700 can include a lid 701 comprising grooves 703, so as to provide a grip for the user to use handling the device. In this way, the device 700 is more ergonomic. In this embodiment, the lid 701 is threadably affixed to the body 704. Specifically, the lid 701 can include female threads (not shown) which engage with male threads (not shown) of the body 704. In this way, the lid 701 is twistable and is removed by way of the user twisting the lid 701 off the body 704. The lid can comprise a carabiner 799 providing a ring, hook, or space, wherein the user's finger can loop around so as to provide a gripping surface for the user when twisting the lid 701. In order to load the applicator device 700 with a container 320 (not shown), the user must twist the lid 701 off and load the applicator device 700 through the first end 711. The applicator device 700 further comprises an hollow arm 775 that is hingedly attached to the lid 701. Specifically, a shoulder portion 779 is integrated onto the exterior of the removable lid 701. Even more specifically, the arm 775 extends from the shoulder portion 779. The arm 775 is configured such that it protrudes from the body 704 when it interfaces therewith. In this embodiment, the arm 775 is rotatable.

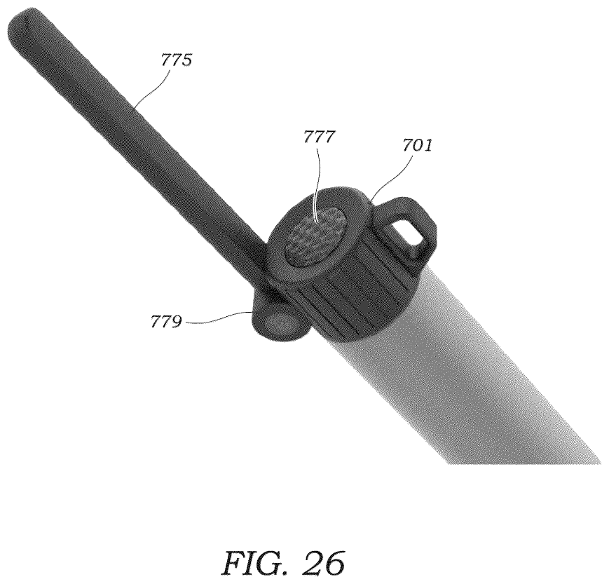

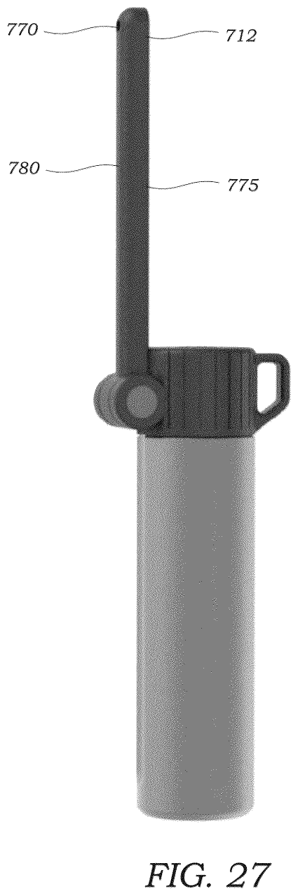

As illustrated in FIG. 26, the arm 775 is able to rotate up to a 1800 angle relative to a longitudinal axis of the shoulder portion 779. As shown in this embodiment, the user pushes down on a button 777 on the top of the lid 701 so as to actuate dispersion of spray from the applicator device 700. As shown in FIG. 27, the arm 775 does not have an appendage extending from the second terminus 712. Rather, the one or more nozzles 770 reside on an interior surface 780 of the arm 775 itself, near or at the second terminus 712.

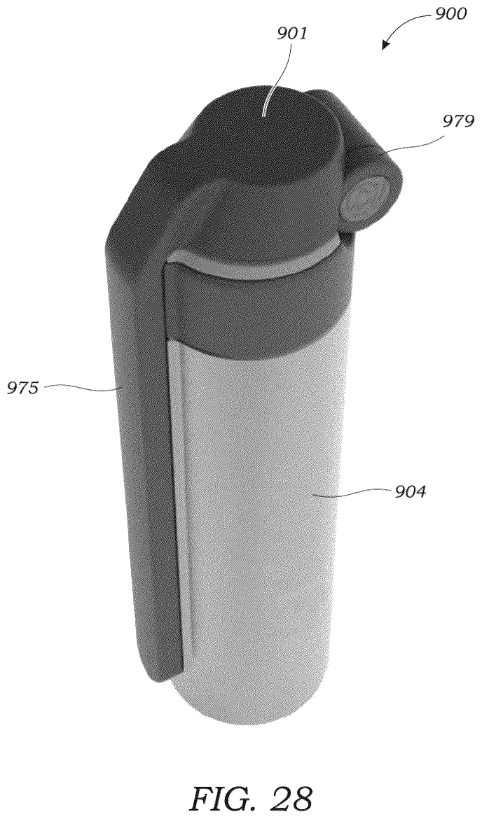

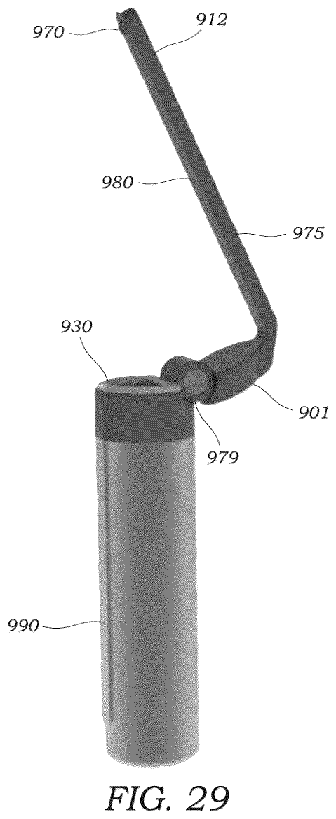

FIGS. 28 and 29 depict an example embodiment of an applicator device 900. As illustrated in FIG. 28, applicator device 900 includes a removable lid 901 that is twistable and removed by way of the user twisting the lid 901 off the body 904. In order to load the applicator device 900 with a container 320 (not shown), the user must twist the lid 901 off and load the applicator device 900 through the first end 911. The applicator device 900 further comprises an hollow arm 975 that is hingedly attached to the body 904. As best shown in FIG. 29, a shoulder portion 979 connects the cover 930 with the lid 901 integrated with the hollow arm 975. Even more specifically, the hollow arm 975 extends from the shoulder portion 979. In this embodiment, the hollow arm 975 is movable along the longitudinal axis of the shoulder portion 979 and can rotate in an upwards direction in the extended configuration. In this embodiment, the body 904 does not have a recess to house the arm 975. Rather, the hollow arm 975 interfaces with a protruding exterior surface 990 of the body 904, and protrudes therefrom when the hollow arm 975 is in the closed configuration. Moreover, in this embodiment, the one or more nozzles 970 reside at the second terminus 912 along the interior surface 980 of the hollow arm 975.

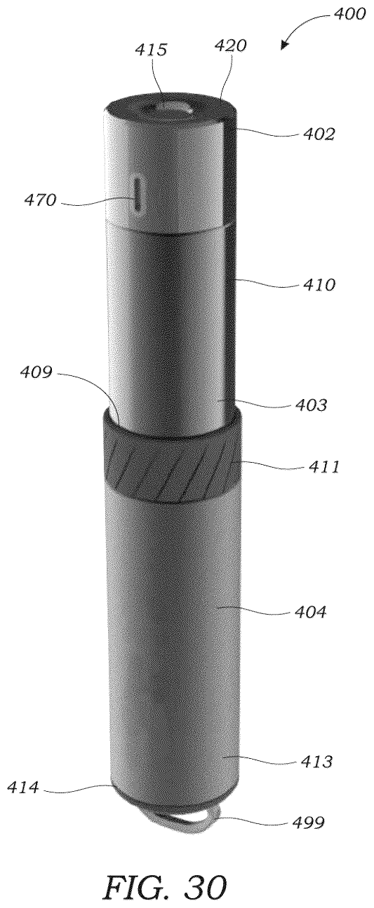

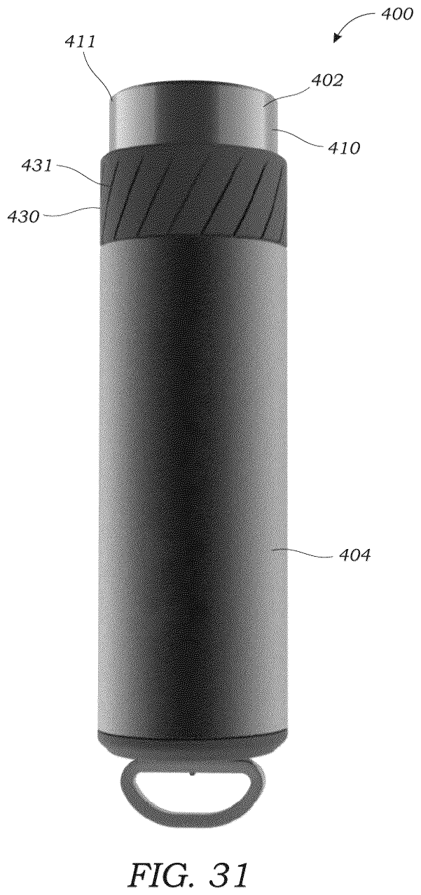

FIGS. 30 and 31 depict an example embodiment of an applicator device 400. As illustrated in FIG. 30, the applicator device 400 does not have an arm or a lid. Rather, the applicator device 400 includes a telescoping assembly comprising an inner telescoping base 410 and an outer telescoping body 404. Further, the inner telescoping base 410 is telescopingly connected to the outer telescoping body 404 so as to slide and/or twist within body 404. Specifically, the outer telescoping body 404 comprises an internal chamber 409 configured to receive the inner telescoping base 410. The telescoping assembly comprises an extended configuration and a closed configuration. In the extended configuration, the inner telescoping base 410 extends out from the first end 411 of the body 404 so as to expose the one or more nozzles 470 embedded thereon. Specifically, a distal end 403 of the inner telescoping base 410 is contiguous and in telescopic connection with the first end 411 of the body 404 when the device 400 is in an extended configuration. More specifically, the inner telescoping base 410 comprises a proximal end 402, wherein the one or more nozzles 470 reside. In this embodiment, the fluid, such as a topical substance, is ultimately sprayed out of the one or more nozzles 470 on the inner telescoping base 410. In this embodiment, the actuator 415 can extend from a top portion 420 of the inner telescoping base 410. In this embodiment, the actuator 415 is configured so as to extend from the top portion 420 along the central axis of the base 410.

In this embodiment, the container (not shown) comprising the fluid, such as topical substance, can be loaded into the receptacle (not shown) of the outer telescoping body 404 through the second end 413. Specifically, the second end 413 comprises a removable cap 414, wherein the removable cap 414 is configured to engage with the body 404 through a press-fit connection, a hinge assembly, or other connecting mechanism determined by one skilled in the art. In some examples, a hinge is provided to connect the cap with the second end. Further, a button can also be provided with the hinge assembly so as to trigger release of the cap 414 hingedly attached to the body 404. In other examples, the cap 414 and body 404 are threadably affixed to one another. In yet other examples, the cap 414 is in press-fit engagement with the body 404 and is removable upon a user exerting a force to pull the cap 414 in a distal direction from the device 400. Further, the removable cap 414 can comprise a carabiner 499 providing a ring, hook, or space, wherein the user's finger can loop around so as to provide a gripping surface for the user when pulling the cap 414 from the body 404.

As illustrated in FIG. 31, in the closed configuration of this embodiment, at least a portion of the inner telescoping base 410 is housed within the outer telescoping body 404. In this configuration, the proximal end 402 of the inner telescoping base 410 is proximal to the outer most edge of the outer telescoping body's first end 411, thereby protruding from the outer telescoping body 404. In this configuration, the surface of the base 410 comprising the one or more nozzles 470 (not shown) is housed within the body 404. In this way, the body 404 functions as a cover for the one or more nozzles 470 (not shown) and prevents inadvertent actuation of the device 400.

The inner telescoping base 410 is slidingly connected to the outer telescoping body 404. In this way, the applicator device 400 can transition between the extended configuration and the closed configuration by way of the user turning the body 404 in a direction opposite of the base 410. Thus, the base 410 can retract or extend as a function of the turning movement. In some examples, the first end 411 of the outer telescoping body 404 comprises a gripping surface 430. In some examples, the gripping surface comprises grooves 431 so as to improve ergonomics. In this manner, the gripping surface 430 provides a grasping area for the user to use to prompt retraction and extension of the base 410 from the body 404.

Both the base 410 and the body 404 comprise an annular or tubular shape. However, those of skill in the art will also appreciate that this embodiment can also comprise other shapes.

This embodiment can further comprise an LED light feature 28 (not shown) so as to provide visual signaling (e.g., if the sunscreen housed within the body requires refilling, light blinking to assist the user in finding a lost or misplaced device, flashlight functions, etc.). Further these described features can also be provided on any of the other applicator device embodiments described herein.

Any of the applicator device embodiments described herein can further comprise a carabiner, a clip, a link, a hook or a fastener of the like. In this way, the applicator device embodiments can attach onto other objects and/or to provide a gripping surface area for the user.

Moreover, the applicator device embodiments described herein can further comprise a bottle opener (not shown). A bottle opener can be integrated with the device 100 or extend therefrom. For example, the bottle opener can form a part of the lid 200 or extend from the body 300.

All of the embodiments described herein can comprise no electronics. In this manner, a user can simply load a container 320 into the device 100 and use the device 100 to spray the topical substance from the container 320 onto a desired location of the body 300, or the like. In some embodiments, the device 100 comprises electronics 21. For example, the device 100 is IoT 63 connected, comprises sensors 44, a battery 24, a PCB 22, USB connector or port 30, Bluetooth, or BLE connectivity, and/or is connectable with a smart device or remote device of the like. In some embodiments comprising electronics 21, the pressure sensor 44 and/or force sensor 44 are proximate to the actuator 15 (e.g., button). Thus, when a user triggers actuation (e.g., presses the button 15), the sensor(s) 44 is activated. The sensor(s) 44 connect to the processor 111, and data is transmitted to the software application (e.g., mobile application, smart watch application, web-based software, etc.) so as to be translated into visual charts and data illustrations that show (1) when a user used the fluid, such as a topical substance (e.g., when the user applied sunscreen); and (2) the duration for which the fluid or topical substance (e.g., sunscreen) has been applied. The data transmitted and processed will then be used to activate reminders, or notify or suggest to the user a certain task (e.g., reapply sunscreen, make an appointment with dermatologist, etc.).

Moreover, the device 100 can incorporate the camera sensor 44 (not shown). In some embodiments, a modular attachment piece (e.g., a “Selfie Stick”) can be affixed to the device 100 so as to enable the user to use an application to take imagery data that can later be used to evaluate skin and skincare. For example, using an application, a user can take a photo of their skin and use it to evaluate their sunscreen coverage.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1-20. (canceled)

21. An applicator device comprising:
a body configured to house a container comprising a fluid, the body further configured to receive the fluid from the container;
one or more sensors;
a flexible tube configured to receive the fluid from the body;
a removable lid attached to the body;
a hollow arm configured to transition between a closed configuration and an extended configuration, the hollow arm comprising one or more nozzles, wherein the one or more nozzles are in fluid communication with the flexible tube, wherein the one or more nozzles are configured to spray the fluid therefrom; and
an actuator configured to cause the fluid to travel from the container into the one or more nozzles and spray therefrom.

22. The applicator device of claim 21, the body further comprising a tubing adaptor, wherein the tubing adaptor comprises a valve inlet in airtight communication with a valve in the container, the valve inlet configured to receive the fluid from the container, the tubing adaptor further comprising a central conduit and a valve outlet, wherein the fluid received from the valve inlet is configured to pass through the central conduit and into the valve outlet.

23. The applicator device of claim 22, wherein the flexible tube comprises a channel, a tube inlet and a tube outlet, wherein the tube inlet is affixed to the valve outlet and is configured to receive the fluid therefrom, wherein the channel is configured to receive topical substance from the tube inlet and deliver the fluid to the tube outlet, wherein the tube outlet is configured to receive the fluid from the channel, wherein the tube outlet is in fluid communication with the one or more nozzles.

24. The applicator device of claim 21, wherein the hollow arm further comprises a first terminus and a second terminus, wherein the first terminus is affixed to the removable lid, the hollow arm further comprising a central channel extending from the first terminus to the second terminus, wherein the central channel is configured to receive at least a portion of the flexible tube.

25. The applicator device of claim 21, wherein the flexible tube is further configured to extend through the body in a proximal direction, and wherein at least a portion of the flexible tube interfaces with the removable lid.

26. The applicator device of claim 21, wherein the body further comprises a base having a cover, wherein the cover comprises a shoulder portion, and wherein the removable lid is hingedly attached to the body by the shoulder portion.

27. The applicator device of claim 21, wherein, the actuator, upon being triggered by a force applied thereto, is configured to exert pressure onto the container, wherein the pressure exerted onto the container is configured to cause the fluid to travel from the container into the one or more nozzles.

28. The applicator device of claim 21, wherein the fluid comprises a topical substance, such as a skincare product, a sunscreen, a tanning oil, a mosquito repellant, a moisturizer, a lotion, a gel, a cream, a serum, a makeup, a soap, a medicinal agent, a drug agent, or an aloe vera.

29. The applicator device of claim 21, wherein the one or more sensors comprise one or more of an ultraviolet sensor, a pressure sensor, a camera sensor, a visible light sensor, a photodiode, an infrared sensor, a temperature sensor, a gyroscope sensor, an accelerometer, an ultrasonic sensor, a tilt sensor, a flex sensor, a touch sensor, a humidity sensor, a flow sensor, a Global Positioning System sensor, an alcohol sensor, and a force sensor.

30. The applicator device of claim 21, further comprising a communication interface and electronics, wherein the electronics are operatively coupled to the one or more sensors and are configured to process signals received from the one or more sensors, wherein the processed signals are configured to transmit to a remote device or application, and wherein the processed signals received by the remote device or application include information relating to a user's monitored skin data and trend information.

31. The applicator device of claim 22, wherein the tubing adaptor further comprises a proximal portion, wherein the actuator is configured to extend from the proximal portion.

32. The applicator device of claim 25, wherein the one or more nozzles reside on the second terminus.

33. The applicator device of claim 21, wherein the body further comprises a receptacle, wherein the receptacle comprises a first end having a removable cap, and wherein upon removal of the cap, the container is configured to be inserted into the receptacle or removed therefrom.

34. The applicator device of claim 33, wherein the removable cap is threadably affixed to a male threaded end of the body.

35. The applicator device of claim 21, wherein the body further comprises a base comprising a housing, and wherein the one or more sensors are disposed in the housing.

36. The applicator device of claim 21, wherein the body further comprises a base with a cover, wherein the cover comprises a cavity, wherein the actuator is configured to protrude in a proximal direction into the cavity and extend therefrom.

37. The applicator device of claim 36, wherein the body further comprises a recess longitudinally extending from the cover to a distal end of the body, and wherein at least a portion of the hollow arm is configured to press-fit interface with the recess.

38. The applicator device of claim 21, wherein the body further comprises a tubing adaptor and a base, wherein the base comprises an opening for receipt of the tubing adaptor.

39. The applicator device of claim 21, further comprising a clip configured to enable the applicator device to attach onto another object.

40. The applicator of claim 21, further comprising a locking mechanism, wherein locking mechanism comprises a first configuration and a second configuration, wherein the locking mechanism is configured to impede accidental triggering of the actuator in the first configuration, and wherein the locking mechanism is configured to enable triggering of the actuator in the second configuration.
